(19) United States
(12) Patent Application Publication
(10) Pub. No.: US 2005/0154270 A1
(43) Pub. Date: Jul. 14, 2005

(54) Application of the kelvin probe technique to mammalian skin and other epithelial structures

(76) Inventors: Richard Nuccitelli, Norfolk, VA (US); Richard Sanger, Woods Hole, MA (US); Peter J.S. Smith, Falmouth, MA (US)

Correspondence Address: REED SMITH, LLP;ATTN: PATENT RECORDS DEPARTMENT, 599 LEXINGTON AVENUE, 29TH FLOOR, NEW YORK, NY, 10022-7650, US

(21) Appl. No.: 11/031,188

(22) Filed: Jan. 7, 2005

Related U.S. Application Data
(60) Provisional application No. 60/534,910, filed on Jan. 8, 2004.

(51) Int. Cl. A61B 5/05

(52) U.S. Cl. 600/345

(57) ABSTRACT

A system and method is disclosed for obtaining measurements of the electric fields around skin wounds and lesions on mammals noninvasively. The system and method is comprised of a vibrating metallic probe tip that is placed close to the skin in the air. By applying a series of known voltages to the metal probe tip or to the skin beneath it, the skin's local surface potential can be measured and the lateral electric field can be calculated from the spatial distribution of surface potential measurements. Surface artifacts that can affect the measurements are removed and active feedback is used to maintain a constant distance between the probe and the skin surface.

### CROSS-REFERENCE TO RELATED APPLICATION

This application claims the benefits of U.S. provisional application Ser. No. 60/534,910, filed Jan. 8, 2004, which is incorporated herein by reference in its entirety.

### FIELD OF THE INVENTION

This application is directed to a method and system for acquiring information from skin and other epithelia. More particularly, this application is directed to the use of this information as a diagnostic tool.

### BACKGROUND OF THE INVENTION

It is known that ionic currents exit skin wounds. The ultimate driving force for all wound currents is the voltage generated across the epidermis. The epidermis of the skin normally generates a voltage across itself, termed the transepithelial potential, by pumping positive ions from its apical to its basal side. FIG. 1a depicts a diagram of a typical epithelial cell exhibiting a polarized distribution of Na⁺ and K⁺ channels. The segregation of Na⁺ channels to the apical end of the epithelial cell and K⁺ channels to its basal end, while utilizing a Na⁺/K⁺-ATPase to lower intracellular [Na⁺] and raise intracellular [K⁺], results in a flow of positive ions across the epithelium. This low intracellular [Na⁺] (combined with the negative membrane potential) results in Na⁺ movement into the cell on the apical end where the channels are localized, and the high intracellular [K⁺] results in K⁺ efflux on the basal side where the K⁺ channels are localized. This transepithelial ion flux creates a transespidermal potential of between 20-55 mV, inside positive, in mammalian skin and has been termed the “skin battery.” Current flow is limited by the very high resistance of the stratum corneum and the tight junctions between epidermal cells that form the multilayers that compose the epidermis.

As can be seen in FIG. 1c, after wounding, this transepidermal voltage will immediately drive current out the low resistance pathway created by the wound. Since this positive wound current flows toward the wound on the basal side of the epidermis, and then away from the wound on the apical side, a lateral electric field will be generated by this flow of wound current on both sides of the epidermis but will exhibit opposite polarities on the two sides. The field at the surface of the wound will have a positive pole near the wound site and deeper in the epidermal multilayer will have the opposite polarity.

The presence of these electric fields can have an effect on would healing. In particular, various studies have shown that the endogenous electrical field near the wound directs epithelial cell migration to improve wound healing. Manipulation of electric fields near the wound could have direct application in enhancing wound healing. However, there has been no consistent methodology established for the use of electric fields in the treatment of wounds. This inconsistency is due to the lack of reliable information regarding the electric fields associated with normally healing wounds in humans. Specifically, the polarity and magnitude of the endogenous wound current within and directly adjacent to the wound must be determined before comprehensive treatments can be formulated. In addition, a demonstration that endogenous wound fields are attenuated in chronic wounds would also be necessary. Standard techniques for determining this information are limited.

One direct method to detect such lateral fields at different depths in the skin is to insert electrode pairs at precise depths to measure the voltage difference between regions lateral to the wound site. Reported measurements of wound lateral fields in (non-human) animals used either glass microelectrodes or micropuncture silver wire electrodes. These methods have several disadvantages.

Both the glass microelectrodes and the chlorided silver wire surfaces are fragile and either break (glass) or are damaged (AgCl) during skin puncture, increasing the risk of the procedure. Electrode tip placement, both the depth and relative lateral spacing, is difficult to reliably reproduce because the electrodes must be positioned using a micromanipulator that is mounted on a support that is usually not directly attached to the subject under study. Therefore, any slight movement by the subject can exert a stress on the electrode, which does not move with the subject. In addition, to reduce noise, the measurement set-up must be placed in an electromagnetically shielded cage, which would severely hamper the portability and ultimate patient utility of the measurement system.

The use of surface skin electrodes also presents problems. These electrodes are placed on the highly resistive stratum corneum while the signal that they must detect is beneath this layer at the stratum granulosum. Due to the variability of the resistance of the stratum corneum from day to day, body location and emotional state, it is difficult to reliably measure very small, potential variations (several millivolts) over small distances on the order of 100 μm.

One indirect method for measuring electric fields in skin utilizes a Kelvin probe. Typically used to measure the work function of various metals, a Kelvin probe functions by creating a parallel plate capacitor with one plate being the probe head and the other being the surface being studied. By regulating a biasing voltage applied to either the probe or the surface being studied, the work function of the surface can be quickly measured. The Kelvin probe has also been used to measure the surface potential of various plant materials. However, this method has several problems when applied to mammals.

Unlike a relatively immobile plant, mammals are prone to constant movement and the mammal must be relatively still while measurements are being taken. While anesthesia can be used to immobilize a subject, this poses unacceptable health risks to humans. Even if the subject is not moving, the skin on mammals is pliable and is subject to constant movement as the mammal breathes and the beating heart circulates fluids throughout its body. This constant movement poses a serious problem for making accurate measurements.

Sources of artifactual signals in mammalian skin make accurate skin surface potential measurements difficult. In particular, hair carries a substantial static charge that will influence surface potential readings. In addition, wounds are often filled with interstitial fluids that have a different work function than the surrounding skin, which will influence a measurement of the electric field.

There is definitely a need for a non-invasive approach that would eliminate the problems associated with these standard techniques. The present invention represents such an approach for detecting electric fields in the skin without contacting the region being studied.

### OBJECTS OF THE INVENTION

It is an object of the invention to provide a novel sensing system and method.

It is also an object of the invention to provide a method and system for acquiring surface potential information from mammalian skin and other epithelia.

It is a further object of the invention to provide a method and system for using surface potential information as a diagnostic tool.

It is a yet further object of the invention to provide an instrument for measuring the surface potential of the skin non-invasively.

It is a yet further object of the invention to provide a method and system for monitoring wound healing.

It is a yet further object of the invention to provide a method and system to diagnose a skin condition or disease such as melanoma, basal cell carcinoma and squamous cell carcinoma.

It is a yet further object of the invention to provide a method and system for measuring the efficacy of skin cosmetics.

These and other objects of the invention will become more apparent from the discussion below.

### SUMMARY OF THE INVENTION

One aspect of the present invention is directed to a probe that will measure the electric fields in and surrounding a wound or skin lesion in a mammal. The probe is comprised of a vibrating metallic probe tip that is placed close to the skin in the air. This eliminates the need for penetrating electrodes or contact electrodes and another advantage is that electromagnetic shielding is unnecessary.

This vibrating probe forms a parallel-plate capacitor with the skin surface. If the surface potential of the metal piece is different from the surface potential of the skin near it, there will be a flow of charge between the two surfaces when they are connected. By applying a series of known voltages (V_{b}) to the metal piece or to the skin, one can quickly determine the voltage value at which there is no current flow between the two surfaces, which value will be equal to the surface potential of the skin at that point. After determining the surface potential at several points in a given region, the electric field between any two points is given by the difference in surface potential at these points divided by the distance between them.

Prior to vibrating the probe tip over the target skin, the skin must be prepared to prevent artifacts that will skew the electric field measurement. For example, when hair is present, that hair must be either removed by physical or chemical means or covered by a uniform dielectric such as a polyvinyl film. This film or other non-conducting dielectric material should also be placed over the wound site to ensure that the surface potential measurement made reflects the electric field near the wound and not the work function differences between skin and any interstitial fluids. The distance between the probe and the skin should be held constant during measurement.

### BRIEF DESCRIPTION OF THE DRAWINGS

The accompanying drawings are included to provide an understanding of the invention and constitute a part of the specification.

FIGS. 1a-c depicts a diagram of mammalian skin and the current that is driven out of the skin by the transepidermal potential when a break in the epidermis occurs or a skin lesion results in a low resistance pathway.

FIG. 2 depicts a schematic diagram of the components of the vibrator assembly and probe head of the Bioelectric Field Imager (BFI) device in accordance with one embodiment of the present invention.

FIG. 3 depicts a schematic diagram showing the electrical connections of the BFI in accordance with one embodiment of the present invention.

FIG. 4 depicts a computer graphic interface for computer-controlled operation of the BFI in accordance with one embodiment of the present invention

FIG. 5A depicts a motorized micromanipulator for scanning the probe over different positions on the skin and maintaining a constant distance between the probe and the skin in accordance with one embodiment of the present invention.

FIG. 5B is a photograph of a prototype portable, hand-held BFI device collecting data from a region of a human hand.

FIG. 6 depicts a typical sequence of steps in performing one method for measuring the electric fields in accordance with one embodiment of the present invention.

FIG. 7 depicts a chart showing the signal detection efficiency versus distance from the skin surface for a probe with a surface area of 0.2 mm².

FIG. 8 depicts a graph showing the variation in peak-to-peak voltage, Vₚₜₚ, against the backing potential, V_{b}, in accordance with one embodiment of the present invention.

FIG. 9 depicts a hairless mouse under inhalation anesthesia being scanned by the BFI probe.

FIGS. 10a-b depict graphics showing a summary of results form the BFI scanning two different types of wounds in the hairless mouse. Numbers above the figure represent average measured electric field values in mV/mm.

FIGS. 11a-b depict charts showing a surface potential scan of the skin by the BFI for an unwounded mouse on the same scale as FIG. 10.

FIG. 12a depicts a photograph of a mouse wound.

FIGS. 12b and 12c depict BFI scans of the mouse wounds shown in FIG. 12a.

FIGS. 12d and 12e represents the probe scan of the same region that was scanned in FIGS. 12b and 12c after topical application of a 1 mM solution of the Na⁺ channel blocker, amiloride, dissolved in phosphate-buffered saline.

FIG. 12f depicts a photograph of a mouse wound.

FIGS. 12g and 12h depict BFI scans of the mouse wounds shown in FIG. 12f.

FIG. 13 depicts BFI scans of a mouse wound over several days as the wound healed.

FIG. 14 depicts BFI scans of a human skin wound.

FIG. 15 is a photomicrograph of a multi-probe array consisting of 8 circular gold sensors that are 0.5 mm in diameter.

FIG. 16 depicts two views of mouse melanoma dissected out 10 days after injection of one million B16 murine melanoma cells under the skin. A: view from outside surface; B: view from underneath the skin after dissecting the skin off of the mouse.

FIGS. 17a and 17b depict a BFI scan of a melanoma nodule 10 days after injection of melanoma cells beneath the skin of a C57BL/6 mouse.

FIGS. 17c and 17d depict a control scan of a neighboring region of skin on the same mouse on the same scale as FIGS. 17a and 17b.

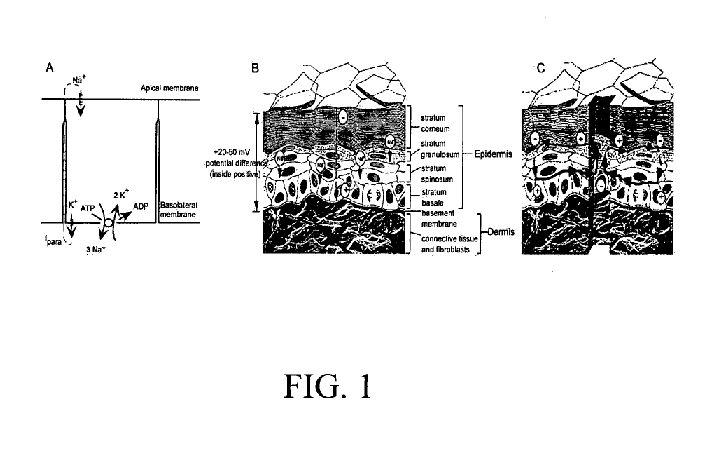

### DETAILED DESCRIPTION OF THE INVENTION

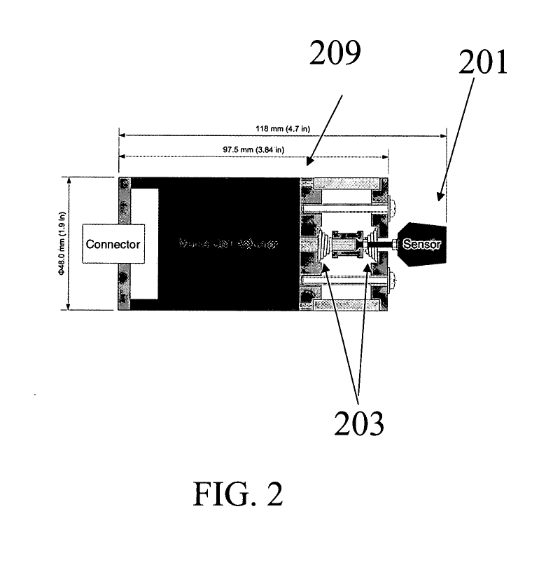

FIG. 2 depicts the probe vibrator and head that is used in one embodiment of the present invention. Probe head 201 is comprised of one or many small metal plates that each have a surface area of 0.2 mm², but a different size can be used and still be within the scope of the present invention. Each plate is connected to a low noise current-to-voltage (I/V) converter housed within the probe head 201 and a copper shield held at the reference potential surrounds the entire head. The vibrator unit 209 is designed to move the probe head along a single axis that is perpendicular to the surface of the skin or epithelia under study. This can be achieved with many types of vibrators based on piezoelectric, magnetostrictive or electromagnetic transducers. One embodiment of the present invention is composed of an cylinder that is 1.9″ in diameter and 4.7″ long and contains an electromagnetic transducer or voice coil actuator, commercially available from BEI Kimco. This suspension system using dual springs 203 stabilizes the axis of vibration and offers a frequency range of 10-400 Hz.

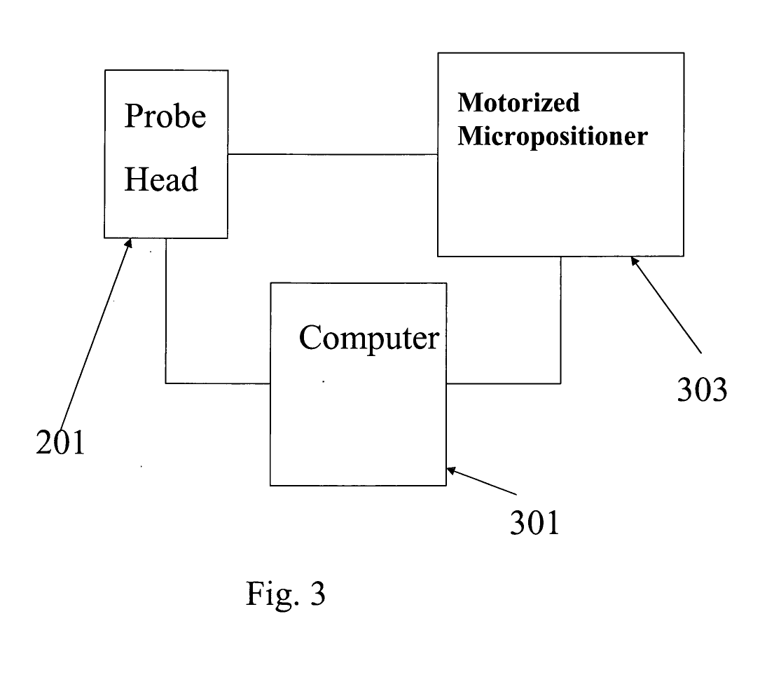

As can be seen in FIG. 3, the probe head 201 is electrically connected to a computer 301 and a motorized micropositioner 303. The computer 301 has an analogue-to-digital converter that is used to acquire signals from the probe and determines the peak-to-peak voltage variation on each current-to-voltage converter connected to each metal sensor as it vibrates near the epithelium. In one embodiment of the present invention, the A/D conversion is performed by a Data Translations A/D converter. Software written in Visual Basic determines the peak-to-peak value of the signal from the probe for different values of V_{b} and averages a specified number of these readings to reduce the contribution of random noise to the signal.

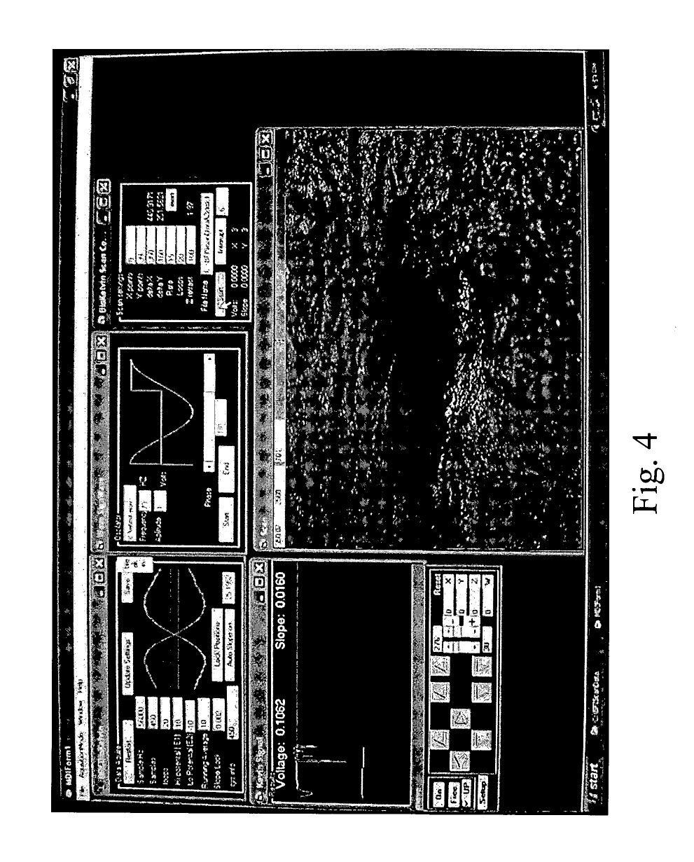

FIG. 4 depicts one embodiment of a graphical interface used on the computer 301. On the upper left corner, the probe real-time output is displayed and several acquisition parameters such as the desired acquisition rate and number of loops to average is entered there. The desired frequency of vibration is entered in the upper center box and the scanning parameters are entered on the upper right box. The time course of both the averaged voltage and slope are displayed on the lower left panel. A photomicrograph of the wound being studied is displayed on the lower right.

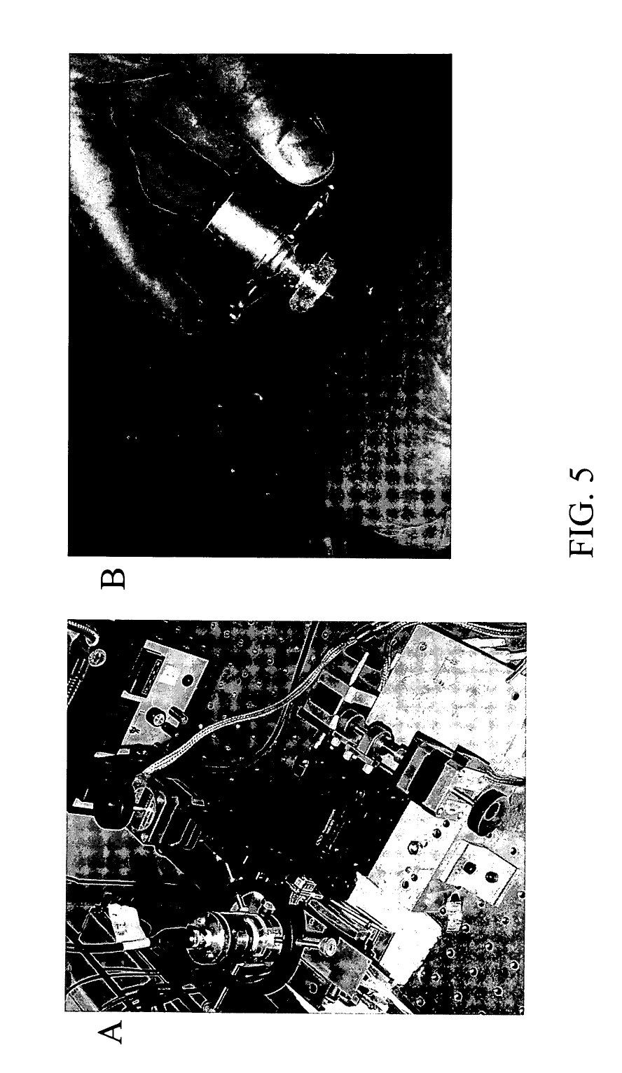

As can been seen in FIG. 3, the motorized micropositioner 303 controls the x-y-z position of the probe head relative to the subject. The motorized positioner 303 is electrically connected to and controlled by the computer 301. The motorized positioner is capable of moving with a 1 μm step size in each direction. FIG. 5a depicts the motorized micromanipulator that controls the x-y-z position of the BFI probe. Although depicted as a probe that is moved via a micropositioner, a probe that is sized to be portable and hand-held is within the scope of the present invention. FIG. 5b depicts such a hand-held probe.

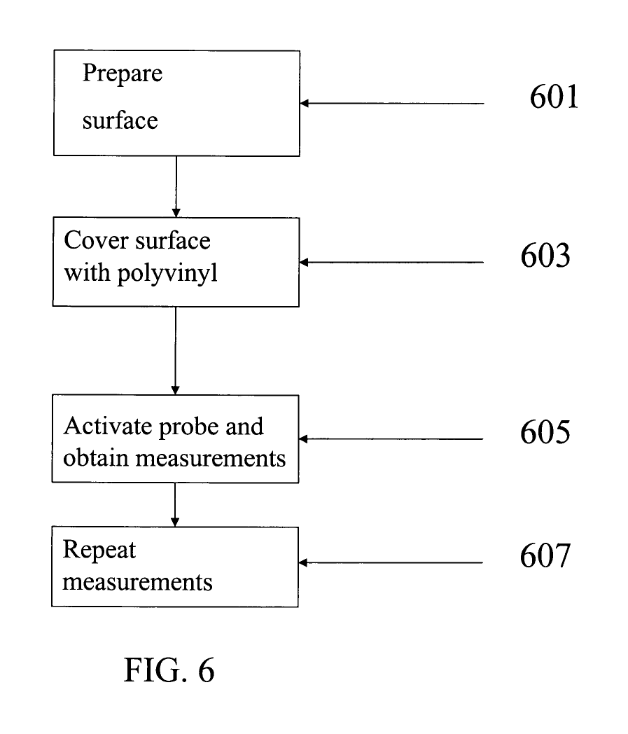

As can be seen in FIG. 6, the method of measuring the electric field near a wound on a mammal is detailed. In step 601, the surface area of the mammal to be measured is prepared. Complete hair removal is important because the static charge on hair influences surface potential readings. That removal can be accomplished by physically shaving the skin and/or using chemical treatments such as Nair. Physically removing the hair with a razor can still leave remnants of hair that can carry charge. The use of chemicals not only dissolves hair but stops further growth for several days. Any possible detrimental effects of chemical treatment on the electrical properties of the skin appear to be negligible.

In step 603, a polyvinyl film can be placed over the wound and must be in very close contact with the skin. If the skin is very dry, a drop of water or mineral oil can be applied to the skin to facilitate this close contact between the skin and the polyvinyl film. This step is taken to eliminate the component of the signal due to work function differences in surface features. The work function refers to the affinity of a given surface for electrons. Every material will have a specific electron affinity. Indeed the first application of the original Kelvin probe was to detect this work function difference between two different metals. Thus, the probe signal is a combination of both the actual surface voltage and the difference in work function between the copper probe tip and the skin surface. Complications can arise when scanning wounds because the interstitial fluid in the wound has a very different work function than that of the surrounding skin and this is detected by the probe. However, if a thin layer of polyvinyl film is placed over the wound, only the work function of the polyvinyl is seen by the probe while the skin surface voltage is detected right through this layer since the polyvinyl is a non-conductor with a dielectric constant of 3.5. This is ideal as it allows the measurement of the electric field near the wound and not the work function differences between skin and fluid. It is important to note that this step is not necessary to obtaining an accurate electric field measurement, but it ensures that the electric field measurement is not affected by the work function differences between the skin and any interstitial fluids in the wound.

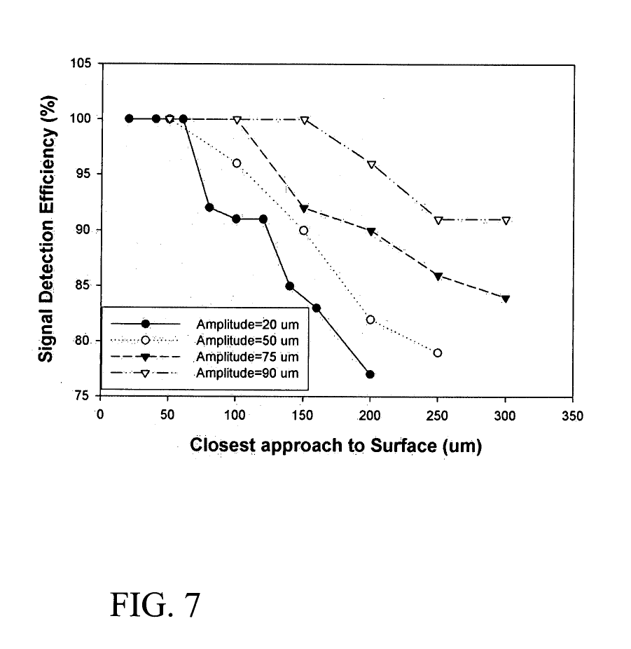

The next step 605 is to activate the probe and begin the measurements. The skin is either grounded during this step or a small voltage of ±5-10 v is applied to it, and the metal probe is vibrated above and normal to the skin. The vibration creates an oscillating current that is converted into an oscillating voltage by the A/D converter in the probe head. The vibration amplitude used is approximately 90 μm or greater. FIG. 7 is a chart that shows the signal detection efficiency of the probe for four different amplitudes. They indicate that a vibration amplitude of 90 μm or greater is required to detect all of the signal with a distance of 150-200 μm between the surface under investigation and the nearest approach of the 0.2 mm² probe. Smaller amplitudes required that the probe be positioned less than 100 μm from the surface for maximum signal detection.

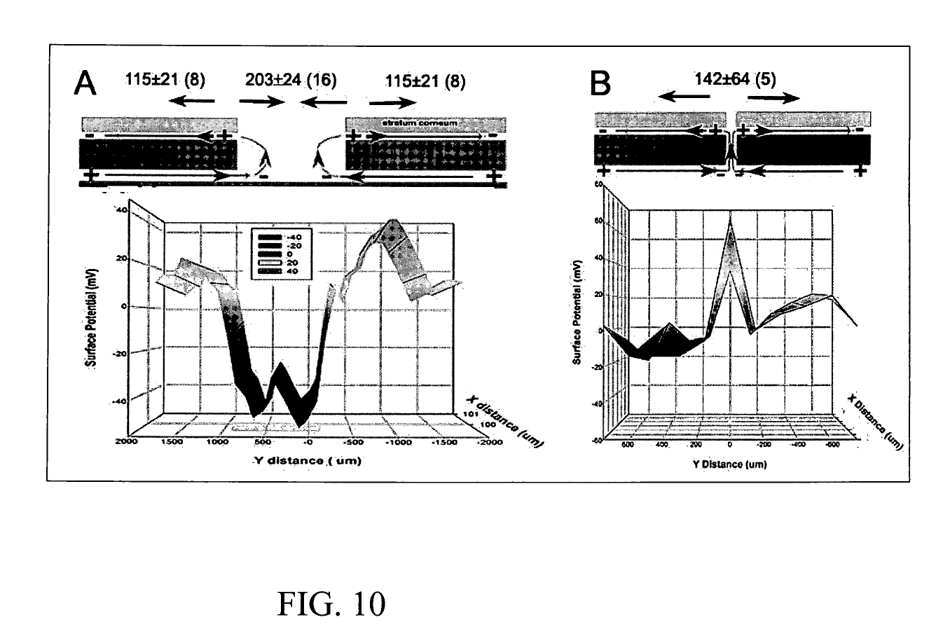

FIG. 7 also illustrates the dependence of signal detection efficiency on the distance of closest approach to the surface being studied. The signal falls off fairly sharply when the gap is larger than 150 μm for a 0.2 mm² probe. Active feedback is incorporated on the height control described below, which is adequate to insure that these two surfaces never touch while the mouse wounds are scanned. Under these conditions, the spatial resolution of the probe is equal to the size of the metal plate used and all of the data collected in FIG. 10-16 used a head with plates that were 320×700 μm. Another probe size that can be used is a circular plate 500 μm in diameter. Since the capacitance is proportional to the surface area of the probe divided by the gap between probe and skin, larger probes would allow a larger gap to be used. Of course, this also reduces spatial resolution so the optimal probe size is dependent on the size of the region of interest. For the 1 mm long wounds described here, the spatial resolution of 320 μm was adequate to generate reproducible electrical field maps.

The probe uses active feedback to control the distance between the probe and the skin and is based on the fundamental theory of the probe. The fundamental principal of this technique is that the skin potential can be measured by vibrating a small, flat piece of metal close to it in air. This forms a parallel-plate capacitor with one plate being the skin surface. If the surface potential of the metal is different from the surface potential of the skin below it, there will be a flow of charge between the two surfaces when they are connected. By applying a series of known voltages (V_{b}) to the probe, the voltage at which there is no current flow between the two surfaces can be quickly determined and that value is equal to the surface potential of the skin just below the probe. This can be rapidly achieved by measuring the Vₚₜₚ when V_{b}=±10 V and then drawing a straight line between these two Vₚₜₚ values. The slope of this line is inversely proportional to the distance between the probe and the skin. This can be seen most clearly from the equation for the output voltage,
Vₒ=(V_{c}+V_{b})GRCω(d/dₒ)sin(ωt+φ)
where d is the oscillation amplitude, dₒ is the average distance between the sample and probe tip, G is the amplifier gain, V_{c} is the voltage difference between the probe and the sample, V_{b} is the voltage applied to the probe, ω is the angular frequency of vibration.

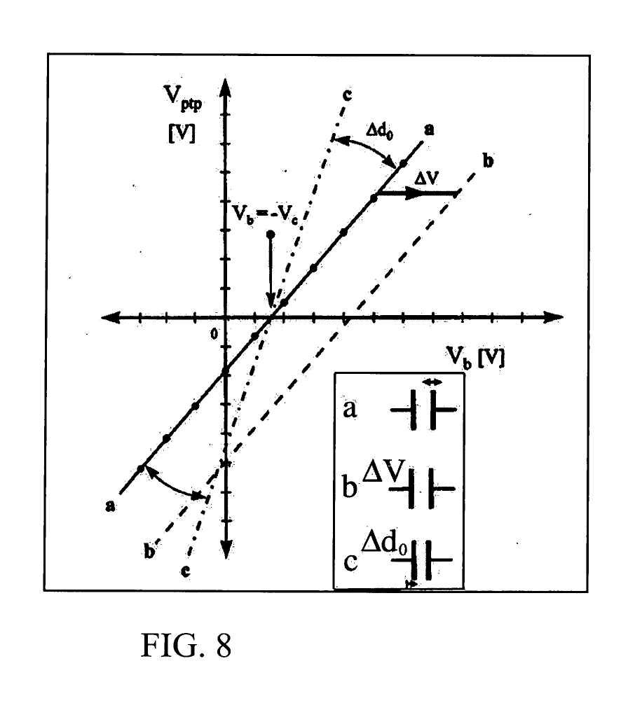

The voltage between the peaks of this sine wave, V_{pyp}=mV_{b}+c where m=2GRCω(d/dₒ). Thus, if Vₚₜₚ is plotted versus V_{b}, it results in a straight line whose slope is inversely proportional to the distance between probe and sample. FIG. 8 depicts a chart showing this relationship. Curve “b” shows the effect of a change in specimen surface potential and “c” shows the slope dependence on the mean spacing. The x axis intercept provides the unknown surface potential where V_{b}=−V_{c}.

This analysis is done by software in real time so that the distance information can be fed back to the z stepper motor to provide a very sensitive method for maintaining a constant spacing between probe and sample. This distance is displayed continuously on the computer monitor as well as on the data output for each scan. This feedback is applied continuously during scanning so that the distance between probe and skin is kept constant.

As can be seen in FIG. 6, step 607 shows that the measurement step 605 is repeated often to average the signal to eliminate noise. In order to reduce the noise to about 1 mV, an average of about 1000 measurements is made. In addition, filter routines are incorporated to reject large noise spikes. This method results in clear measurements of the electric fields surrounding wounds in mammals.

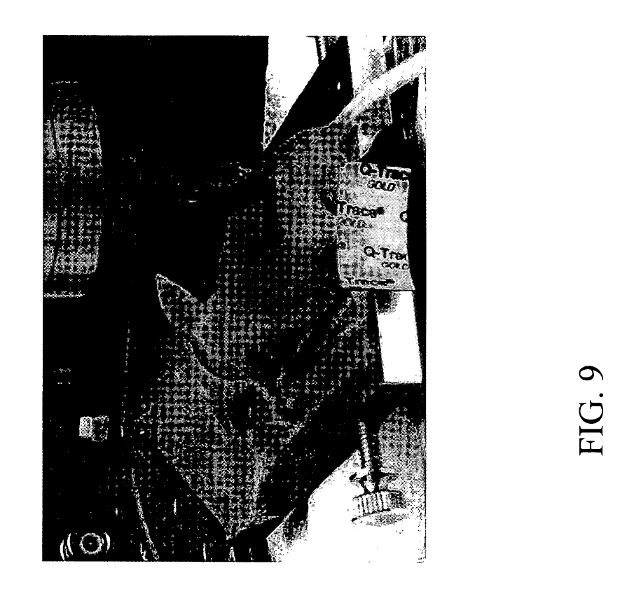

Comprehensive testing was performed on mice. FIG. 9 depicts a hairless mouse under isoflurane inhalation anesthesia being scanned by the probe. Electrical contact is made with the skin of the mouse by using a conductive plastic Q-trace tab on its foot. FIGS. 10a and 10b depict a summary of results observed on mouse wounds with the indicated mean electric field values plus or minus SEM in mV/mm and the number of mice studied given in parentheses. FIG. 10a shows the surface potential measurements where there is a significant break in the epidermis. As indicated by the scale, the average field measured over the wound is approximately 200 mV/mm and the field reverses and falls to 115 mV/mm at the edge of the wound. FIG. 10b depicts the measured electric field when the wound does not exhibit a large break in the epidermis and this value is typically 142 mV/mm. In addition, it can be seen that the surface potential for the smaller wound is positive around the wound. In contrast, the larger wound shows a positive surface potential around the edges of the wound and a large negative surface potential for the area within the wound. This occurs because the larger gap in the epidermis exposes the relatively negative region beneath the epidermis where the current is flowing in the opposite direction toward the wound.

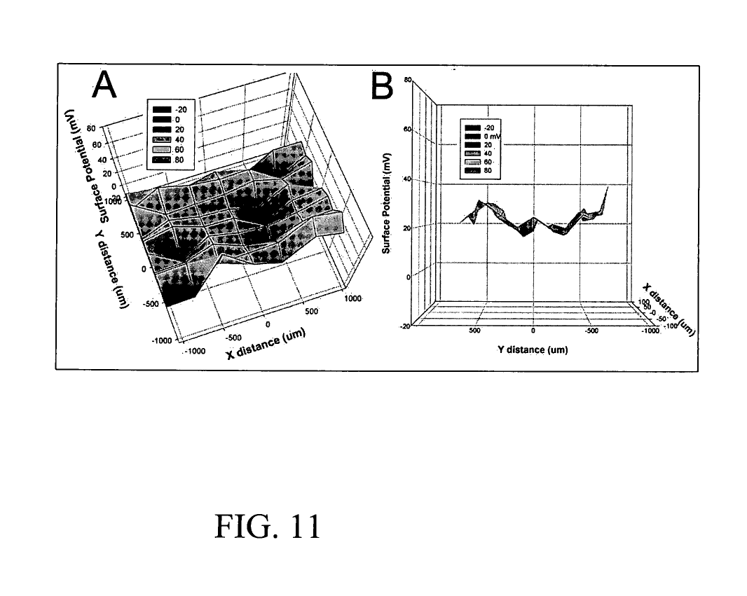

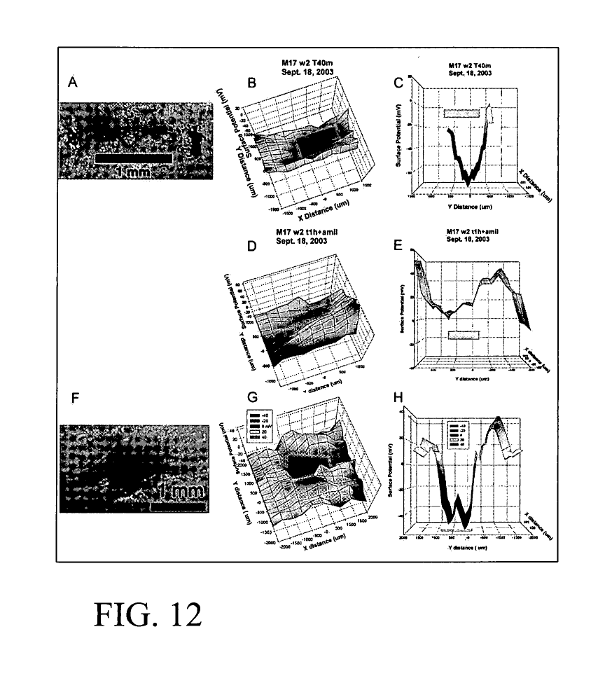

FIGS. 11 and 12 depicts the results of a probe scan before and after wounding. FIG. 11a shows a 3-dimensional plot of the skin surface potential prior to a wound being inflicted. FIG. 11b is a two-dimensional cross section view of a 200 μm wide strip from A along the y axis at x=0. FIG. 12 depicts the surface potential distribution after the wound has been inflicted. FIG. 12A and FIG. 12F are micrographs of the two wounds inflicted on different mice with the respective BFI scans of these wounds shown to the right of each micrograph (FIGS. 12B-C and 12G-H). FIG. 12D-E shows the surface potential distribution of the same region scanned in FIG. 12B-C just after the topical application of the Na⁺ channel blocker, amiloride. The 50% reduction in the electric field following amiloride application supports the hypothesis that this electric field is generated by the transepithelial potential that is in part dependent on Na⁺ influx into the epidermis.

As can be seen in FIG. 11, a scan of unwounded skin generally reveals a fairly uniform surface potential with a maximum variation of about 60 mV prior to wounding. However, immediately following wounding, an electric field is detected in the skin around the wound (FIGS. 12B,C). The region over the wound is usually negative with respect to the surrounding skin by 200 mV/mm. The magnitude of this field is dependent on Na⁺ influx because it is reduced by an average of 60±11% (SEM, n=6) when a 1 mM solution of amiloride in saline is added topically to the wound (FIG. 12d,e). In addition to the clearly negative region directly over the wound, a surface potential gradient of 115±64 mV/mm is usually found from the edge of the wound outward. This is generated by the current flowing between the stratum comeum and the stratum granulosum.

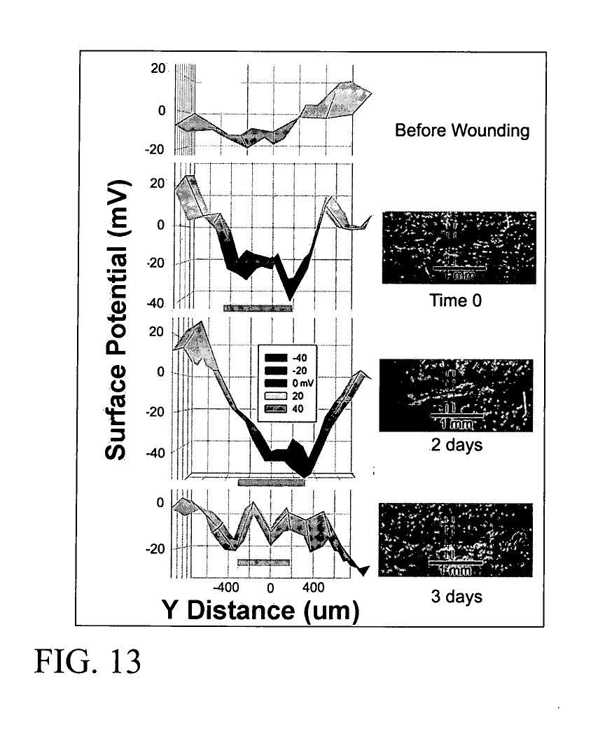

The magnitude of these skin wound electric fields diminishes over the course of time. FIG. 13 depicts scans from the probe for a wound over the course of three days. As can be seen by the decrease in the electric field measured at three days after wounding when the wound appears to be nearly completely healed, there is a positive correlation between the two.

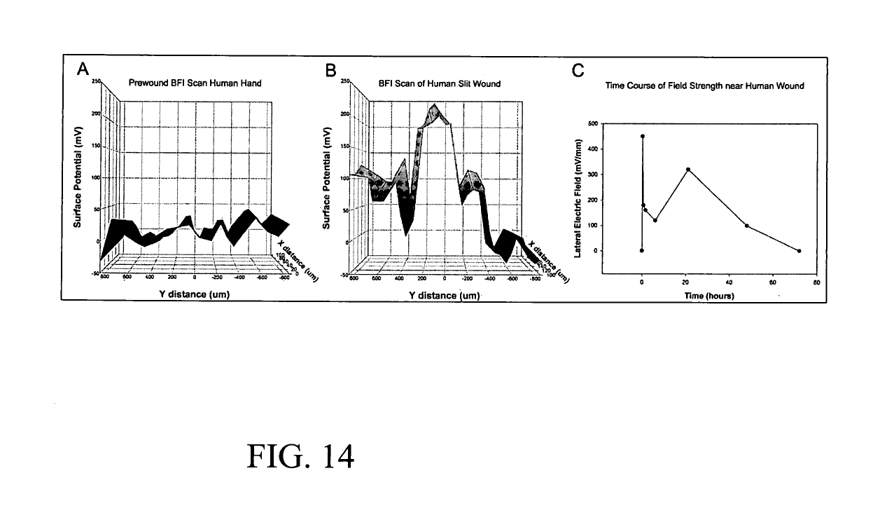

FIG. 14 shows the results from a BFI scan performed on a narrow cut in the hand of a human. The electric field magnitude and time course of healing was similar to that observed on the mouse wounds. This technique could be useful for providing a quantifiable measure of the rate of wound healing as well as determining if there is a correlation between the magnitude of the electric field near human skin wounds and the rate of wound healing.

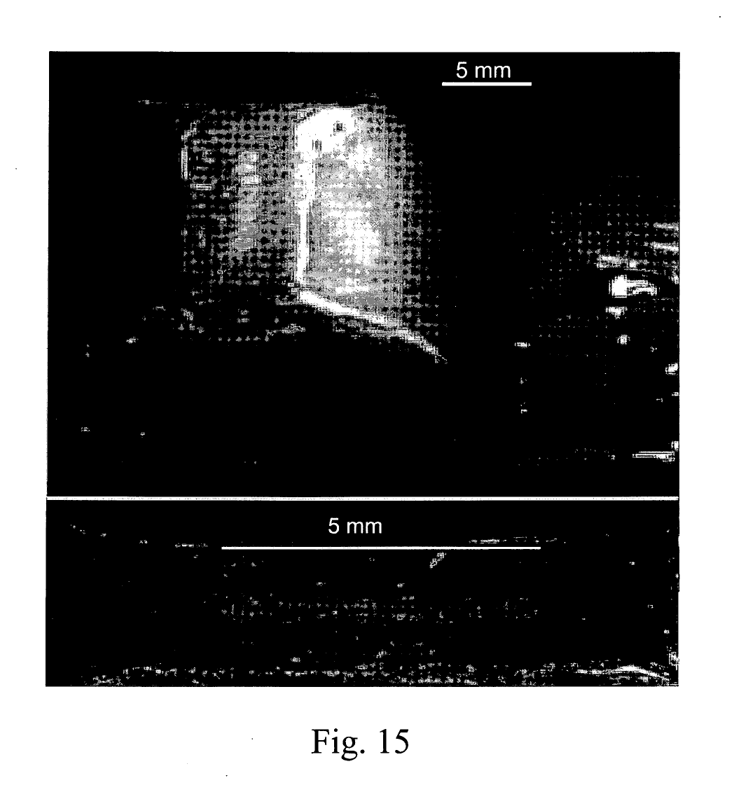

While all of the data collected on mice has been generated by scanning a single probe sensor over the skin in a two-dimensional grid pattern with the aid of a micromanipulator, spatial information can be also obtained without scanning by using an array of sensors. FIG. 15 shows one such array in which 8 sensors are arranged in a linear fashion and makes possible the simultaneous acquisition of the surface potential data from 8 points at once. This shortens the data acquisition time 8-fold while providing electric field information along one axis. Eight A/D converters are located within the head and these are connected to 8 inputs of a Data Translations A/D card capable of digitizing all 8 signals simultaneously at 100 kHz.

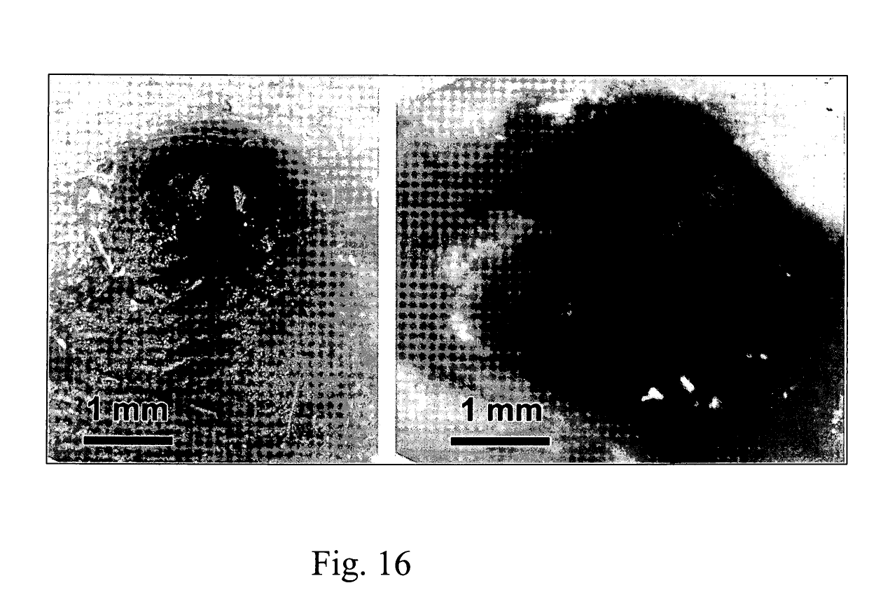

The BFI device can be used to measure the skin surface potential around melanomas in mouse skin. These melanomas can be generated by injecting approximately 1 million B16 murine melanoma cells just beneath the skin in a mouse. FIG. 16 illustrates the appearance of a typical melanoma at 10 days after injection. The left micrograph shows a view from outside of skin while the right micrograph shows a view from underneath the skin on the same scale showing that the melanoma is actually larger than it appears from the apical side.

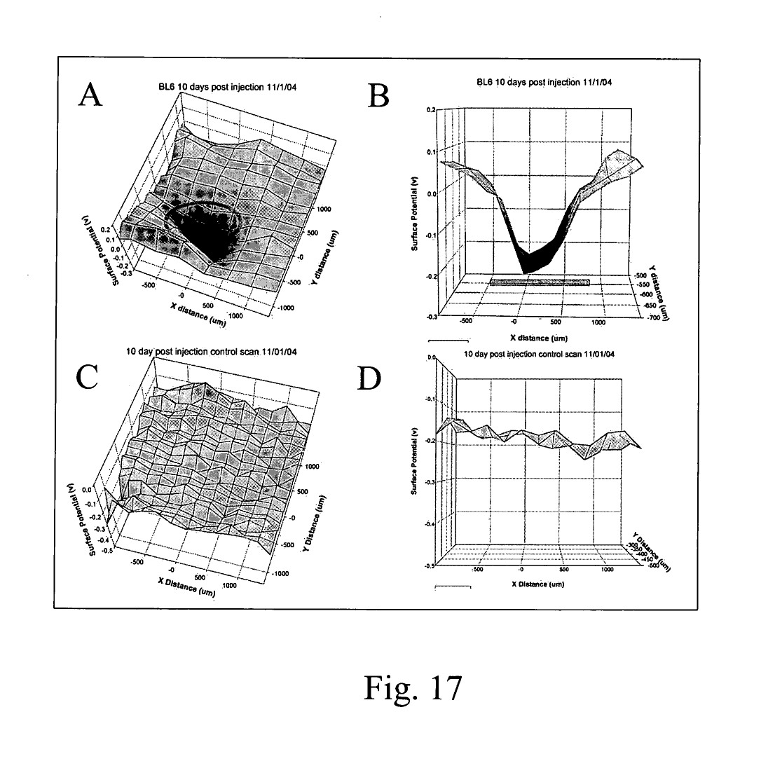

FIGS. 17a-d illustrates the surface potential distribution of one such melanoma in a C57BL/6 mouse. The circular red line in FIG. 17a and the broad line on the x-axis in FIG. 17b indicate the tumor position relative to the scan. This can be compared to the two lower panels that represent a control scan of a nearby region of skin on the same mouse. These data show that the BFI device may be useful for the detection and diagnosis of skin cancers such as melanoma, basal cell carcinoma and squamous cell carcinoma.

The present invention is not to be considered limited in scope by the preferred embodiments described in the specification. Additional advantages and modifications, which readily occur to those skilled in the art from consideration and specification and practice of this invention are intended to be within the scope and spirit of the following claims:

1. A method for evaluating an electric field associated with an epithelium of a mammal comprising the steps of:
positioning a probe a constant distance above the epithelium to create a capacitance between said probe and the epithelium;
applying a bias voltage to said probe or the epithelium;
vibrating said probe; and
measuring the current generated during vibration of said probe to determine the surface potential of the epithelium.

2. The method for evaluating an electric field associated with the epithelium as recited in claim 1, further comprising the step of preparing the epithelium in response to work function artifacts.

3. The method for evaluating an electric field associated with the epithelium of the mammal as recited in claim 2, wherein said preparing step comprises removing hair from the epithelium.

4. The method for evaluating an electric field associated with the epithelium of the mammal as recited in claim 2, wherein said preparing step comprises covering the epithelium with a non-conducting material.

5. The method for evaluating an electric field associated with the epithelium of the mammal as recited in claim 1, further comprising the step of moving the probe laterally along the epithelium to scan an area of the epithelium while maintaining the constant distance between said probe and the epithelium.

6. The method for evaluating an electric field associated with the epithelium of the mammal as recited in claim 5, further comprising the step of graphically displaying the measurements taken in said measuring step.

7. The method for evaluating an electric field associated with the epithelium of the mammal as recited in claim 6, wherein said displaying step comprises a three-dimensional display.

8. The method for evaluating an electric field associated with the epithelium of the mammal as recited in claim 6, wherein said displaying step comprises a graph of the measurements over time.

9. The method for evaluating an electric field associated with the epithelium of the mammal as recited in claim 1, wherein said positioning step utilizes a feedback circuit to maintain the distance between said probe and the epithelium.

10. The method for evaluating an electric field associated with the epithelium of the mammal as recited in claim 1, further comprising the steps of positioning a second probe over the epithelium at the same distance that said probe is maintained; vibrating said second probe; and measuring the current generated during vibration of said second probe to determine the surface potential of the epithelium.

11. The method for evaluating an electric field associated with the epithelium of the mammal as recited in claim 1, wherein the epithelium contains a wound and the electric field evaluated is associated with the wound.

12. The method for evaluating an electric field associated with the epithelium of the mammal as recited in claim 1, further comprising the step of diagnosing the condition of the epithelium based on the electric field associated with the epithelium.

13. The method for evaluating an electric field associated with the epithelium of the mammal as recited in claim 1, further comprising the step of detecting epithelium lesions by measuring the lateral electric field that these lesions generate.

14. The method for evaluating an electric field associated with the epithelium of the mammal as recited in claim 3, wherein the hair is chemically removed.

15. The method for evaluating an electric field associated with the epithelium of the mammal as claimed in claim 11, further comprising the step of quantifying the healing of wounds by monitoring the current over time.

16. A noninvasive diagnostic system for evaluating an electric field associated with an epithelium of the mammal comprising:
a probe comprising a conducting plate;
a vibrating unit attached to said probe for vibrating said probe over the epithelium of the mammal;
a voltage supply for creating a voltage bias between the probe and the epithelium;
a positioning device attached to the probe to maintain a constant distance between the epithelium and the probe; and
a meter for measuring the current generated by the vibrating probe.

17. The noninvasive diagnostic system for evaluating an electric field associated with the epithelium of the mammal as recited in claim 15 further comprising a means for removing work function artifacts on the epithelium.

18. The noninvasive diagnostic system for evaluating an electric field associated with the epithelium of the mammal as recited in claim 17, wherein said probe and said vibrating unit are contained in a handheld housing.

19. The noninvasive diagnostic system for evaluating an electric field associated with the epithelium of the mammal as recited in claim 17, wherein said positioning motor utilizes a feedback circuit to maintain a constant distance between the epithelium and the probe.

20. The noninvasive diagnostic system for evaluating an electric field associated with the epithelium of the mammal as recited in claim 16 further comprising an analog to digital converter.

21. The noninvasive diagnostic system for evaluating an electric field associated with the epithelium of the mammal as recited in claim 16 wherein said means for removing work function artifacts on the epithelium comprises of a non conducting material to cover the epithelium.

22. The noninvasive diagnostic system for evaluating an electric field associated with the epithelium of the mammal as recited in claim 16 wherein said positioning motor is capable of laterally moving the probe across an area of the epithelium while maintaining the constant distance.

23. A method for evaluating an electric field associated with the epithelium of a mammal, comprising the steps of:
removing work function artifacts from the epithelium;
positioning a probe above the epithelium;
applying a voltage to create a bias between said probe and the epithelium;
vibrating said probe; and
measuring the current generated during vibration of said probe.
